(19) United States
(12) Patent Application Publication
(10) Pub. No.: US 2026/0027519 A1
(43) Pub. Date: Jan. 29, 2026

(54) CONTAMINATED AIR TREATMENT

(71) Applicant: I/O ELECTRICAL & MECHANICAL LIMITED, Cootehill, Co. Caven (IE)

(72) Inventor: Shane O'REILLY, Cootehill, Co. Caven (IE)

(21) Appl. No.: 18/997,082

(22) PCT Filed: Jul. 18, 2023
(86) PCT No.: PCT/EP2023/069912

§ 371 Date: Jan. 19, 2025

(30) Foreign Application Priority Data
Jul. 19, 2022 (GB) 2210566.2
Jul. 19, 2022 (GB) 2210571.2

(51) Int. Cl. B01D 53/78; A61L 9/14; B01D 53/34; B01D 53/72

(52) U.S. Cl. CPC B01D 53/78; A61L 9/145; B01D 53/346; B01D 53/72; A61L 2209/111; A61L 2209/14; B01D 2257/406; B01D 2258/06

(57) ABSTRACT

Contaminated air treatment system, comprising: at least one enclosed space for containing a source of contaminated air; a subterranean treatment tunnel comprising one or more openings connecting the treatment tunnel with the enclosed space, said subterranean treatment tunnel having located therein contaminated air treatment means; air circulating means configured to drive contaminated air from the at least one enclosed space into the treatment tunnel via the one or more openings and through the air treatment means, said treatment means comprising at least a first treatment stage comprising a spraying arrangement configured in use to spray the contaminated air to entrap contaminant particles in a liquid/sludge residue, and a treated air exhaust to exhaust treated air after it has passed through the treatment tunnel.

### TECHNICAL FIELD

The present invention relates to systems for treating contaminated air, particularly, but not exclusively, for treating air contaminated by rearing animals in animal enclosures.

### BACKGROUND

Many industrial and agricultural processes give rise to high levels of contaminated air. For example, organic compounds from the waste produced by animals, such as pigs or chickens, reared in enclosures gives rise to such high concentrations of contaminated air that operators of such facilities are often mandated to treat the air before it is released into the surrounding environment.

Commonly, this treatment is undertaken by elevated air scrubbers, typically mounted on the roof, gable wall or upper wall of an enclosure. Such elevated air scrubbers draw in contaminated air from within the enclosure, treat the air, and then exhaust treated air out into the surrounding environment.

However, the use of such elevated air scrubbers is expensive. The cost of the scrubbing units themselves is typically high and their elevated mounting location means they are expensive to install and often difficult (and thus expensive) to maintain and repair. Furthermore, in many settings elevated air scrubbers typically necessitate a high-level of circulation of the contaminated air in the space that contains the source of contaminated air. For example, in a typical animal enclosure with an air scrubber mounted in an elevated position, contaminated air from effluent pits is often drawn up through an animal enclosure before being treated. This can increase the levels of harmful gases, such as ammonia, within the enclosure reducing the welfare of the animals.

A further undesirable aspect of conventional elevated air scrubbers is the fact they typically use toxic chemicals as purifiers.

In a related problem to increase crop yields, it is necessary for soil to be fertilized. Animal slurry is a good fertiliser as it is typically widely available in agricultural settings and reduces reliance on chemical fertilisers. During fertilising operations, animal slurry is added to the soil. Previously, animal slurry was simply spread on the soil to be fertilised. Nowadays, however, to reduce its polluting effects, animal slurry is more commonly added to soil by an injection process where it is injected directly into the soil.

Whilst a good fertiliser for the reasons mentioned above, slurry from animals is a source of emissions that are considered harmful to the environment, in particular ammonia. In many jurisdictions, increasingly strict environmental regulations require ammonia reducing acid to be added to slurry before it is used in fertilising operations. This typically requires the machinery used for fertilising operations to be equipped with tanks of acidified water (typically diluted nitric acid) and means to add acidified water from these tanks to the slurry as it is being injected into the soil.

As well as making fertilizing operations generally more complicated and expensive, for normal agricultural workers, such tanks of acidified water can be awkward to work with and can potentially give rise to serious hazards, particularly if the production of the acidified water requires large quantities of concentrated acid solutions to be stored and mixed.

### SUMMARY OF THE INVENTION

In accordance with a first aspect of the invention, there is provided a contaminated air treatment system. The contaminated air treatment system comprises: at least one enclosed space for containing a source of contaminated air; a subterranean treatment tunnel comprising one or more openings connecting the treatment tunnel with the enclosed space, said subterranean treatment tunnel having located therein contaminated air treatment means; air circulating means configured to drive contaminated air from the at least one enclosed space into the treatment tunnel via the one or more openings and through the air treatment means, said treatment means comprising at least a first treatment stage comprising a spraying arrangement configured in use to spray the contaminated air to entrap contaminant particles in a liquid/sludge residue, and a treated air exhaust to exhaust treated air after it has passed through the treatment tunnel.

Optionally, said treatment tunnel is located at substantially the same level as a base region of the at least one enclosed space.

Optionally, the treatment tunnel runs at least a part of the length of the at least one base region.

Optionally, the one or more openings are positioned along the length of the tunnel adjacent to the at least one base region.

Optionally, the contaminated air treatment system comprises two enclosed spaces, wherein said treatment tunnel is located between the base regions of each enclosed space.

Optionally, the openings comprise a first set of openings positioned along the length of the tunnel on a first side of the tunnel adjacent the first base region of the first enclosed space and a second set of openings positioned along the length of the tunnel on a second side of the tunnel adjacent the second base region of the second enclosed space.

Optionally, each opening of the first set of openings is positioned adjacent to a corresponding opening of the second set of openings, thereby inducing a turning effect in the air in the treatment tunnel in use.

Optionally, each opening comprises a louver comprising at least one air-flow control slat which pivots about a lower edge to control an opening amount.

Optionally, the base region or each base region comprises an effluent pit.

Optionally, the one or more openings are adjustable to control an amount of contaminated air entering the treatment tunnel, thereby enabling a speed of the air in the treatment tunnel to be controlled.

Optionally, the contaminated air treatment system further comprises a pressure sensor and an actuator, said pressure sensor located in the treatment tunnel and configured to send a pressure senor signal to the actuator, said actuator coupled to the one or more openings and configured to control an opening amount of each of the one or more openings responsive to the pressure senor signal from the actuator.

Optionally, the actuator is configured to control the one or more openings to maintain a predetermined air speed in the treatment tunnel.

Optionally, the air treatment means comprises a second treatment stage comprising a spraying arrangement configured in use to spray the air from the first stage with an odorant.

Optionally, the air circulating means comprises a fan bank comprising one or more fans located in the treatment tunnel.

Optionally, the fan bank is positioned between the first treatment stage and the second treatment stage.

Optionally, the treatment tunnel further comprises a waste tank to collect the liquid/sludge residue.

Optionally, the treated air exhaust is located to exhaust the treated air at ground level.

Optionally, a final filtration stage is located on an outlet side of the treated air exhaust to further filter the treated air.

Optionally, the final filtration stage comprises a filtration compound made from wood and/or bark chippings.

Optionally, the at least one enclosed space comprises at least one vent opening the at least one enclosed space to the outside environment, wherein said air circulating means is configured to draw fresh air in through the at least one vent thereby ventilating the at least one enclosed space.

Optionally, the at least one enclosed space is an animal enclosure for housing animals.

In accordance with a second aspect of the invention, there is provided an animal housing facility comprising one or more animal enclosures forming enclosed spaces and a contaminated air treatment system according to the first aspect of the invention.

In accordance with embodiments of the invention, a contaminated air treatment system is provided which comprises a subterranean treatment tunnel. The subterranean treatment tunnel is connected to an enclosed space (for example an animal enclosure) containing a source of contaminated air which is to be treated before being released. The treatment tunnel comprises treatment means (for example one or more spraying systems) configured to treat the contaminated air, and air circulating means which extracts the contaminated air from the enclosed space into the treatment tunnel and drives the contaminated air through the treatment means.

Advantageously, by providing contaminated air treatment means in a subterranean tunnel, there is no need for expensive elevated air scrubbers. Moreover, in many settings, locating the air treatment means in a subterranean tunnel provides a number of further advantages. For example, in animal housing facilities, sources of contaminants arising from the breeding of animals and animal waste are normally collected and retained at, or below, ground level in slurry pits (effluent pits). Treating contaminated air from such sources in a subterranean tunnel, (rather than, for example, by an elevated air scrubber), means the contaminated air is circulated less and is extracted for treatment at the point where it is usually most concentrated.

Furthermore, by locating the contaminated air treatment means in a subterranean tunnel, by-products of the treatment process (for example liquid residue from a spraying system) can be easily pumped back into the effluent pits and, in certain settings, extracted for further processing.

Further still, treatment tunnels in accordance with embodiments of the invention can be readily constructed during the construction of a facility, for example an animal housing facility, thereby providing a permanent means to treat contaminated air arising from the facility.

Similarly, treatment tunnels in accordance with embodiments of the invention can be readily fitted to existing structures/buildings.

Various aspects and features of this invention are defined in the claims.

In accordance with a technique for treating waste, there is provided a facility comprising: an effluent pit in which waste from the facility collects, and a spraying system. The spraying system is configured to spray acidified water in situ onto waste collected in the effluent pit via spraying means to reduce harmful emissions being emitted to the atmosphere from the collected waste.

Optionally, the facility is an animal housing facility for housing animals, and waste from animals housed in the facility collects in the effluent pit. The spraying system is configured to spray acidified water onto waste collected in the effluent pit via spraying means to reduce ammonia emissions from the collected animal waste when the animal waste is subsequently used as crop fertiliser.

Optionally, the spraying system further comprises an acidified water storing means configured to stored acidified water to be sprayed onto the waste collected in the effluent pit via the spraying means.

Optionally, the spraying system further comprises a flow control device for controlling flow of the acidified water from the acidified water storing means to the spraying means.

Optionally, the facility further comprises a control unit for controlling operation of the flow control device.

Optionally, the control unit is configured to control the flow control device so that the acidified water is sprayed onto the waste collected in the effluent pit periodically.

Optionally, the control unit is configured to control the flow control device so that the acidified water is sprayed onto the waste collected in the effluent pit periodically for a predetermined duration.

Optionally, the facility further comprises a gas sensor located in the vicinity of the effluent pit and configured to generate a sensor signal indicative of a concentration of one or more types of gas in the vicinity of the effluent pit and to communicate a corresponding sensor signal to the control unit. The control unit is configured to control the flow control device to so that the acidified water is sprayed onto the waste collected in the effluent pit if the sensor signal indicates that the concentration of one or more gases in the vicinity of the effluent pit has exceeded a predetermined level.

Optionally, the spraying system further comprises means to replenish the acidified water storage means with water from a water supply after acidified water has been sprayed onto waste collected in the effluent pit, and a pH monitoring unit for monitoring the pH of acidified water in the acidified water storing means and an acid pump. The pH monitoring unit is configured to control the acid pump to pump acid from acid storage means into the acidified water storage means if the pH of the acidified water drops below a predetermined pH level.

Optionally, the predetermined pH level is approximately between 3 and 5.5.

Optionally, the acidified water storage means comprise a circulating pump to mix the water from the water supply with the acid from the acid storage means.

Optionally, the acidified water storage means comprises a fluid storage tank.

Optionally, the spraying means comprises one or more nozzles.

Optionally, the spraying means comprises an array of nozzles.

Optionally, the facility is animal housing facility and the array of nozzles are located underneath a grated floor of the animal housing facility through which waste from animals housed in the facility passes.

In accordance with a further aspect of the technique for treating waste, there is provided a method of treating waste. The method comprises: collecting waste from a facility in an effluent pit forming part of the facility, and spraying the collected the effluent pit, in situ, with acidified water.

Optionally, the step of collecting waste comprises collecting animal waste from animals housed in an animal housing facility in an effluent pit forming part of the animal housing facility, and the method comprises using the sprayed waste to produce fertiliser.

In accordance with a further aspect of the technique for treating waste, there is provided a spraying system for incorporating in a facility comprising an effluent pit in which waste from the facility collects. The spraying system comprises spraying means for spraying acidified water onto waste collected in the effluent pit.

In accordance with the technique for treating waste, a facility including one or more effluent pits is provided with a spraying system which is configured to spray waste collected in the one or more effluent pits with acidified water to reduce emissions. Treating collected waste at the point it is collected (in situ) is advantageous because it reduces harmful emissions from the waste as it is collected; and reduces the requirement to subsequently treat the collected waste.

In accordance with the technique, an otherwise conventional animal enclosure facility including an effluent pit for collecting animal waste from animals housed in the facility is adapted to include a spraying system for spraying acidified water on animal waste that has collected in the effluent pit.

This arrangement means that slurry for fertiliser formed from the animal waste collected in the effluent pit is treated with acidified water in situ at the point at which it is produced/collected. When the animal waste collected in the effluent pit is then subsequently used in fertilising operations, it is already treated either reducing or eliminating the requirement to add acidified water to the slurry during, or directly before, fertilising operations. Consequently, this reduces or eliminates the requirement of machinery used in fertilising operations to be provided with awkward and potentially hazardous acidified water storage tanks; and, more generally by minimising or eliminating the requirement for acidified water to be added during or directly before fertilizing operations, the cost and complexity of such fertilizing operations can be reduced.

Furthermore, advantageously, by adding acidified water to animal waste as it is collected, the emissions to the atmosphere of harmful gases including ammonia from the effluent pit is reduced. This improves the welfare of animals housed in the animal enclosure and reduces the pollution produced by the facility.

Further still, by adding acidified water to animal waste that is formed by diluted nitric acid, the fertilizing effect of slurry produced from the animal waste is improved because of the prolonged exposure of the animal waste to the nitrogen component of the acidified water.

Advantageously, spraying systems in accordance with examples of the technique for treating waste can be readily retrofitted to existing facilities (such as animal enclosures) or can be easily incorporated in new facilities (such as animal enclosures) as they are being constructed.

Various aspects and features of this technique are defined in the claims.

### BRIEF DESCRIPTION OF THE DRAWINGS

Embodiments of the present invention will now be described by way of example only with reference to the accompanying drawings where like parts are provided with corresponding reference numerals and in which:

FIGS. 1 and 2 provide simplified schematic diagrams depicting an animal housing facility comprising a contaminated air treatment system arranged in accordance with certain embodiments of the invention;

FIGS. 3 and 4 provide simplified schematic diagrams providing a more detailed view of a treatment tunnel arranged in accordance with certain embodiments of the invention, and

FIG. 5 provides a simplified schematic diagram depicting a cross-section of a treatment tunnel arranged in accordance with certain embodiments of the invention.

FIG. 6 provides a simplified schematic diagram providing an exploded view depicting the positioning of a spraying system within an animal facility in accordance with certain examples of a technique for treating waste

FIG. 7 provides a simplified schematic diagram depicting in more detail components of a spraying system in accordance with the technique for treating waste.

### DETAILED DESCRIPTION

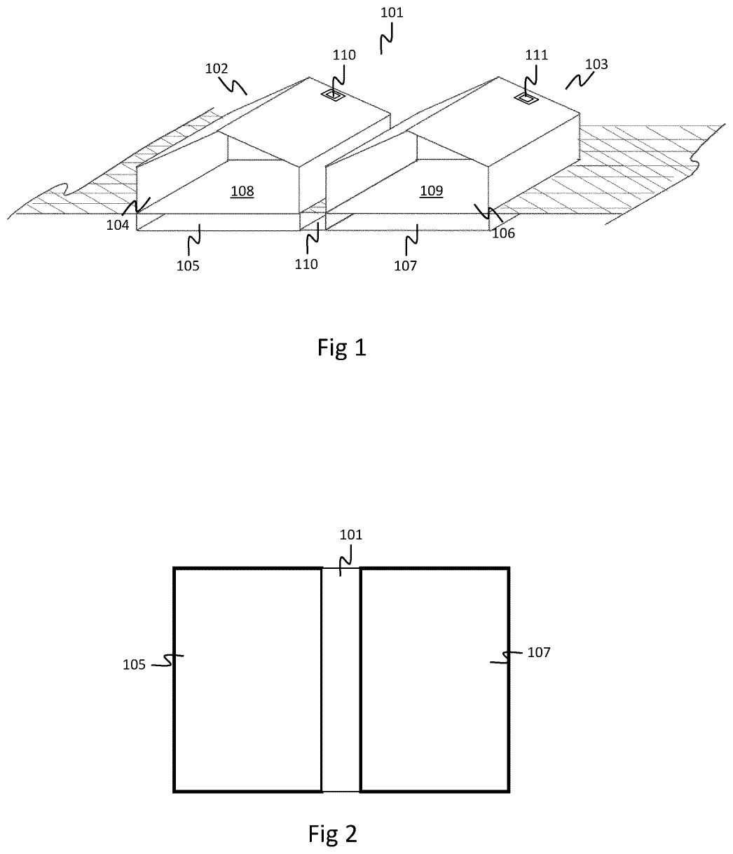

FIGS. 1 and 2 provide simplified schematic diagrams depicting an animal housing facility 101 comprising a contaminated air treatment system arranged in accordance with certain embodiments of the invention. The contaminated air treatment system is configured to extract contaminated air from the animal housing facility (i.e. ventilate the facility), preventing the build-up of harmful gases and particles within the animal housing facility that would be detrimental to the welfare of the animals enclosed therein, and to treat the extracted air to an extent that the treated air can be released into the surrounding environment. This is achieved without the use of elevated air scrubbers which are conventionally used for these purposes.

The animal housing facility 101 comprises a first enclosed space provided by a first housing unit 102 and a second enclosed space provided by a second housing unit 103. The first housing unit 102 comprises a first animal enclosure 104 and first effluent pit 105 located in a base region of the first housing unit 102 and the second housing unit 103 comprises a second animal enclosure 106 and a second effluent pit 107 located in a base region of the second housing unit 103.

In use, animals, for example pigs, cows or chickens, are kept in the animal enclosures 104, 106. The air in the animal enclosures 104, 106 is contaminated by organic contaminants emanating from the animals themselves (gases, fluid particles and dust particles) and the air in the effluent pits 105, 107 is contaminated by contaminants (including gases such as ammonia and fluid particles) emanating from the effluent stored therein.

Each animal enclosure is typically provided by a self-contained building comprising walls and a roof and includes feeding, heating and lighting facilities for the animals enclosed in the animal enclosures 104, 106.

A ventilation system serves each housing unit 102, 103 which is described in more detail below.

The effluent pit 105, 107 of each housing unit 102, 103 is positioned directly beneath a grated floor 108, 109 of the animal enclosure 104, 106. The grated floors 108, 109 comprise grates (not shown) allowing waste from animals kept in the housing units 102, 103 to pass into the effluent pits 105, 107.

The grated floors 108, 109 are substantially at ground level, whilst the effluent pits 105, 107 are below ground level. Located between the effluent pits 105, 107 is a treatment tunnel 110. The treatment tunnel 110 is located at substantially the same level as the effluent pits 105, 107 (i.e., subterranean). As will be described further below, the treatment tunnel forms an integral part of the ventilation system of each housing unit 102, 103.

As can be seen from FIG. 2, the treatment tunnel 110 runs substantially the length of each effluent pit 105, 107.

Advantageously, this arrangement is simple to construct when erecting the animal housing facility 101 as it simply requires an additional trench (for the treatment tunnel 110) to be excavated at the same level as the effluent pits 105, 107. Moreover, by locating the treatment tunnel underground, space around the animal housing facility 101 is made available for other purposes for example to locate other equipment and/or to provide more room for people, animals and vehicles to move around the facility.

The treatment tunnel 110 can be formed using any suitable construction technique for the construction of sub-ground-level structures. For example, the treatment tunnel 110 may be formed by lining a suitably dug trench with concrete, for example reinforced concrete, and then forming a suitable roof over the top of the lined trench. Such lining will include suitably formed apertures for the openings (described further below) which connect the treatment tunnel 110 to the enclosed space or spaces containing the source of contaminated air. As will be understood, such a technique also requires suitable apertures to be formed in the walls of the structure forming the enclosed space for containing the source of contaminated air. These apertures can be formed at the time of constructing the structure forming the enclosed space (for example if the treatment tunnel is being constructed at the same time as the enclosed space) or after construction of the structure forming the enclosed space (for example if the treatment tunnel is being retrofitted to an existing structure).

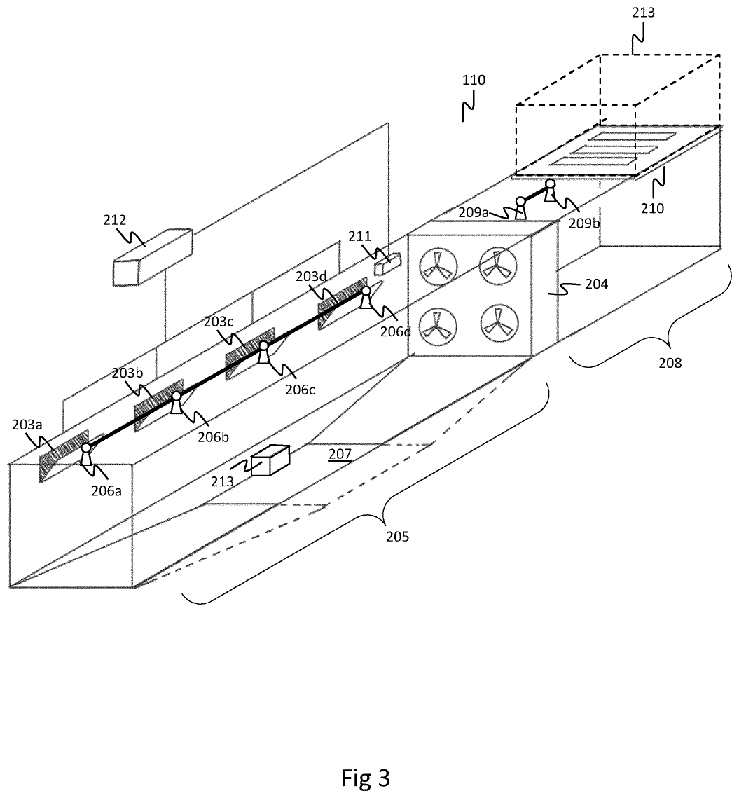

FIGS. 3 and 4 provide simplified schematic diagrams providing a more detailed view of the treatment tunnel 110 in accordance with certain embodiments of the invention.

The treatment tunnel 110 comprises two walls 201, 202 each of which is adjacent to one of the effluent pits 105, 107. Along the length of these walls 201, 202 there are plurality of intake louvers 203a, 203b, 203c, 203d, 203e, 203f, 203g, 203h which provide openings that connect the treatment tunnel 110 directly to the effluent pits 105, 107 and connect the treatment tunnel 110, via the effluent pits 105, 107 to the animal enclosures 104, 106. Louvers on either side walls are positioned opposite to each other, forming a plurality of opposite-facing pairs of louvers along the length of the treatment tunnel 110.

In this embodiment, the treatment tunnel 110 is directly adjacent to the effluent pits 105, 107, thus the intake louvers 203a, 203b, 203c, 203d, 203e, 203f, 203g, 203h provide direct openings to the respective effluent pits 105, 107. However, in alternative embodiments, the treatment tunnel 110 may not be directly adjacent to the effluent pits 105, 107, therefore the intake louvers 203a, 203b, 203c, 203d, 203e, 203f, 203g, 203h may provide openings to one or more suitable conduits (ducting) which connect the treatment tunnel 110 to the effluent pits and connect the treatment tunnel 110, via the effluent pits to the animal enclosures 104, 106.

Positioned along the treatment tunnel 110 is a fan bank 204. The fan bank 204 provides air circulating means that gives rise to a negative pressure in the treatment tunnel 110 relative to the effluent pits 105, 107 and animal enclosures 104, 106 of the housing units 102, 103. This drives contaminated air from the effluent pits 105, 107 and contaminated air from the animal enclosures 104, 106, into the treatment tunnel.

The animal enclosures 104, 106 each comprise one or more vents 110, 111 opening the inside of each housing unit 102, 103 to the outside environment. Operation of the fan bank 204 also gives rise to a negative pressure in the effluent pits 105, 107 and animal enclosures 104, 106 relative to the outside environment. This draws air from the surrounding environment into the animal enclosures 104, 106 via the one or more vents 110, 111. In this way, the air circulating means (e.g. the fan bank 204 in the example shown in FIG. 3) that drives contaminated air into the treatment tunnel also serves to draw in fresh air from outside the animal enclosures 104, 106, thus forming an integral part of the ventilation system of each housing unit 102, 103.

The size and configuration of the fan bank 204 and the speed at which it operates can be selected based on factors such as the ventilation requirements of the enclosed spaces, the dimensions of the tunnel and the desired air speed through the tunnel (discussed in more detail below). In one exemplary embodiment, the fan bank 204 comprises four 92 cm diameter fans configured to operate at 1400 rpm. However, in other embodiments, a fan bank comprising a different number of fans and/or of a different diameter and/or operating at a different speed can be used.

In alternative embodiments, the fan bank or banks (or any other suitable air circulating means) can be positioned in any other alternative location that gives rise to a negative pressure in the treatment tunnel 110 and the animal enclosures 104, 106 relative to the outside environment to drive contaminated air into the treatment tunnel 110.

In use, contaminated air is drawn in through the louvers 203a, 203b, 203c, 203d, 203e, 203f, 203g, 203h by the fan bank 204. This contaminated air is initially treated by a first treatment stage 205 located in the region of the treatment tunnel 110 on the intake side of the fan bank 204 and then treated by a second treatment stage 208 located on the outtake side of the fan bank 204.

The first treatment stage 205 is provided by a high-pressure misting system which consists of a plurality of high-pressure misting nozzles 206a, 206b, 206c, 206d which each project a high-pressure liquid mist into the treatment tunnel 110. Each misting nozzle 206a, 206b, 206c, 206d is positioned in the vicinity of each opposite-facing pair of louvers.

The liquid mist projected into the treatment tunnel 110 typically comprises a mixture of water and surfactant (which reduces the surface tension of the droplets of the liquid mist). Droplets of the liquid mist entrap contaminant particles in the contaminated air to form a liquid/sludge residue which collects in a waste tank 207 located at the bottom of the treatment tunnel 110 along the length of the first treatment stage 205.

Air drawn through the fan bank 204 is drawn into the second treatment stage 208 located on the outtake side of the fan bank 204. The second treatment stage 208 consists of a second plurality of high-pressure misting nozzles 209a, 209b which each project a second high-pressure liquid mist into the treatment tunnel 110. The second high-pressure liquid mist typically consists of water, a surfactant and an odorant to perfume the air.

Air treated by the second treatment stage 208 is driven out of the treatment tunnel 110 via an exhaust provided by an outtake louver 210. In the example shown in FIG. 3, the outtake louver 210 is positioned at ground level. However, in other embodiments, the outtake louver can be positioned at other levels. For example, an exhaust stack can be provided which rises up from the end of the treatment tunnel and the outtake louver is positioned on this exhaust stack, thereby exhausting treated air at a higher level (i.e. above ground level).

In certain embodiments, a final filtration stage can be included, for example on the outlet side of the outtake louver 210. For example, as depicted in FIG. 3 by broken lines, a filtration block 213 can be positioned on top of the outtake louver 210 comprising a filtration compound made, for example, from wood and/or bark chippings. Treated air exiting the outtake louver 210 passes through the filtration block 213 which absorbs contaminants in the air exiting the treatment tunnel 110.

By virtue of the treatment provided by the first and second treatment stage and, in certain embodiments, the filtration block 213, the concentration of contaminants (and thus, for example, foul odours) in the air exiting the treatment tunnel 110 is reduced.

Further, because the fan bank 204 draws fresh air into the housing units 102, 103, the concentration of harmful gases (for example ammonia) and particles is reduced within the animal enclosures 104, 106.

In other embodiments, other numbers of treatments stages and other sequences of treatment stages can be used. For example, the tunnel can alternatively include more than two treatments stages or a single continuous treatment stage.

Tanks in which the liquid for the liquid mist is stored and pumping equipment for pressurising the liquid can be located in any suitable location, for example at ground level allowing easy access for replenishment and maintenance.

Typically, each intake louver comprises a moveable slat which is adjustable to control the size of the opening provided to the relevant effluent pit. The fan bank 204 typically operates at a constant speed, therefore the size of the opening provided by the louvers 203a, 203b, 203c, 203d, 203e, 203f, 203g, 203h determines the speed of the air traveling through the treatment tunnel 110.

The speed of the air moving through the treatment tunnel 110 is an important consideration. It must be fast enough to provide adequate ventilation to the animal enclosures above, but not so fast that the contaminated air moves through the treatment tunnel 110 too rapidly and therefore does not undergo sufficient treatment.

To control the speed of air moving through the treatment tunnel 110, a pressure sensor 211 is provided which is located within the treatment tunnel 110. The pressure sensor 211 measures the air pressure within the treatment tunnel 110 (which is directly related to the air speed in the treatment tunnel 110). The pressure sensor 211 generates a pressure sensor signal indicative of the detected air pressure which is communicated via a suitable signal line to an actuator 212. The actuator 212 is mechanically connected to each of the louvers 203a, 203b, 203c, 203d, 203e, 203f, 203g, 203h via suitable mechanical linkages and by virtue of this arrangement can control the position of the moveable slat of each louver and correspondingly, the size of the opening provided by each louver.

The actuator 212 includes a control unit which is configured to operate the actuator 212 in response to the pressure sensor signal. The control unit of the actuator 212 is typically configured to control the actuator 212 to actuate the position of the slats of the louvers to maintain a predetermined air speed. Specifically, if the air pressure signal corresponds to an airspeed above the predetermined threshold speed, the control unit is configured to control the actuator 212 to move the position of the slats of the louvers to reduce the size of the openings provided to the effluent pits and housing facility air. Conversely, if the air pressure signal corresponds to an airspeed below the predetermined threshold speed, the control unit is configured to control the actuator 212 to move the position of the slats of the louvers to increase the size of the openings provided to the effluent pits and housing facility air inlets.

The air speed in the treatment tunnel 110 that the actuator 212 is configured to maintain will depend on factors such as the dimensions of the treatment tunnel, the temperature of the tunnel and the ventilation requirements of the enclosed spaces containing the sources of contaminated air. In a typical example, the actuator 212 is configured to maintain an airspeed in the treatment tunnel 110 of approximately 2 m/s. However, in other examples this may be a slower air speed and in other examples this may be a faster air speed.

Typically, the treatment tunnel 110 operates continuously. That is, the fan bank 204 continually draws contaminated air in via the effluent pits 105, 107 which are continuously treated by the first treatment stage 205 and second treatment stage 208; treated air is continuously exhausted from the outtake louver 210, and fresh air is continually drawn into the housing units 102, 103 via the vents 110, 111.

A pump unit 213 is located in the treatment tunnel 110 which is arranged to pump the liquid/sludge residue that has collected in the waste tank 207 into the effluent pits 105, 107 via suitable conduits (not shown) connecting the waste tank 207 to the effluent pits 105, 107.

Alternatively or additionally, the liquid/sludge residue can be extracted from the (for example using the pump unit 213) treatment tunnel to separate storage means (for example one or more suitable tanks) for disposal or further processing.

In certain embodiments, the effluent pits 105, 107 themselves have located therein treatment means that periodically (for example 4 times every 24 hours) treat the effluent. Such treatment means typically comprise a spray system configured to spray an ammonia reducing liquid (for example acidified water) onto the effluent in the effluent pits 105, 107.

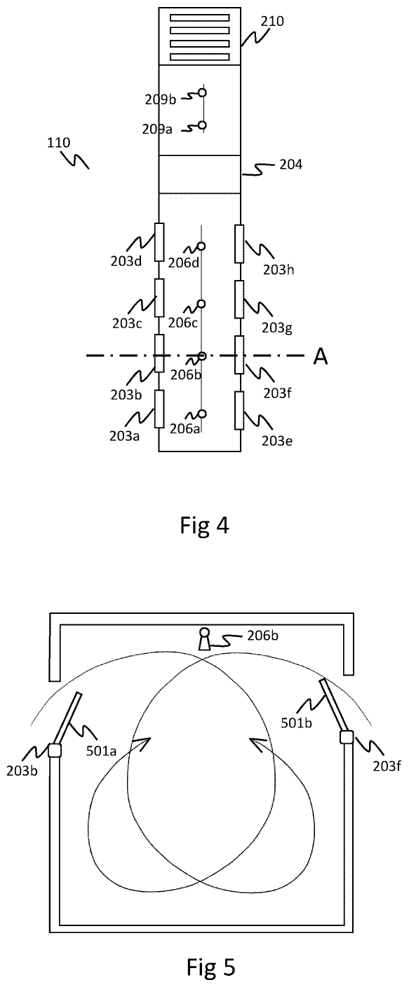

As described above, in certain examples, louvers on either side walls are positioned opposite to each other. The louvers on either side wall can be directly opposite each other (as shown in FIG. 4), forming a plurality of opposite-facing pairs of louvers along the length of the treatment tunnel 110. Alternatively, rather than directly facing each other, the louvers on either side wall can be staggered. Advantageously, positioning louvers on either side wall opposite to each other (either directly opposite or in a staggered arrangement) induces a turning of the air in the first treatment stage 205 which increases the degree to which contaminated air drawn in through the louvers interacts with the bonding liquid and increases the extent to which particles in the air are captured in the liquid/sludge residue. This turning effect is described further with reference to FIG. 5.

FIG. 5 provides a simplified schematic diagram depicting a cross-section of the treatment tunnel 110 taken along line A shown in FIG. 4. As can be seen from FIG. 5, air is drawn in from opposite-facing pairs of louvers 203b, 203f. As can also be seen from FIG. 5, each louver comprises an air control slat 501a, 501b which pivots about a lower edge to control the amount the louver is open.

The two separate flows of air drawn through these opposite-facing pairs of louvers 203b, 203f interact with each other forming a spiral which “turns” the air along the first treatment stage 205. This turning effect is induced by each opposite-facing pairs of louvers along the length of the first treatment stage 205 of the treatment tunnel 110.

Advantageously, because the treatment tunnel 110 is positioned adjacent to the effluent pits 105, 107, heat from the effluent pits and animal enclosures 104, 106 will warm the treatment tunnel 110. This warming increases the temperature of the air being treated in the first treatment stage 205 and second treatment stage 208 which increases the efficacy of the treatment of the contaminated air.

In a typical embodiment, by virtue of the proximity of the treatment tunnel 110 to the effluent pits 105, 107, the temperature in the treatment tunnel 110 can be maintained at an adequate temperature to optimise the treatment processes (for example approximately 22 to 24 degrees Celsius). However, in certain examples, the proximity of the treatment tunnel to the effluent pits and the animal enclosures may not give rise to adequate heating to optimise the treatment processes. Accordingly, in certain examples, the treatment tunnel itself may be provided with a heating system, for example a hot water pipe running adjacent the intake louvers.

In a typical arrangement, various monitoring sensors are located in each of the treatment tunnel 110, effluent pits 105, 107 and animal enclosures 104, 106. These sensors may include CO2 sensors, ammonia sensors and temperature sensors. Sensor signals from these sensors are communicated via suitable signal lines to a suitable location, for example a control room, or, via a suitable wired or wireless link, to a remote location, where conditions in the animal housing facility 101 can be monitored.

Effluent and liquid residue from the treatment tunnel 110 can be periodically emptied from the effluent pits 105, 107 and disposed of and/or used for other purposes, for example as a nitrate source for the production of organic fertilizers.

In the embodiment described above, the treatment tunnel is positioned between two housing units and directly adjacent one external wall of the effluent pit of each housing unit. However, embodiments of the invention can be manifested in any suitable way in which a subterranean treatment tunnel is located relative to one or more animal housing units in such a way that contaminated air is driven from the animal housing units through contaminated air treatment means located in the treatment tunnel.

For example, in embodiments in which an animal housing facility comprises a single housing unit (rather than two adjacent housing units as shown in FIG. 1) a treatment tunnel in accordance with certain embodiments of the invention may located adjacent a single effluent pit and thus contaminated air will only enter the tunnel from one side.

Conveniently, the treatment tunnel may be positioned along an external peripheral side of an animal enclosure (as shown in FIG. 1) enabling the treatment tunnel to take the simple form of a straight tunnel. However, in certain embodiments, a treatment tunnel may extend further around the periphery of an animal enclosure, for example around two or more sides. Further, in animal housing facilities comprising non-straight (e.g., curved) walls, the treatment tunnel may be shaped to conform according with such non-straight walls.

Contaminated air treatment systems in accordance with embodiments of the invention can be used in settings other than those relating to the rearing of animals in enclosures. For example, contaminated air treatment systems in accordance with certain embodiments of the invention can be used to treat contaminated air arising from other sources such as sources of contaminated air is sewage treatment, food production and waste (refuse) management facilities.

As will be understood, the length and size (height and width) of a treatment tunnel arranged in accordance with embodiments of the invention will typically be determined by factors including the dimensions and ventilation requirements of the enclosed space or enclosed spaces to which it is connected and the size and configuration of air treatment means located therein.

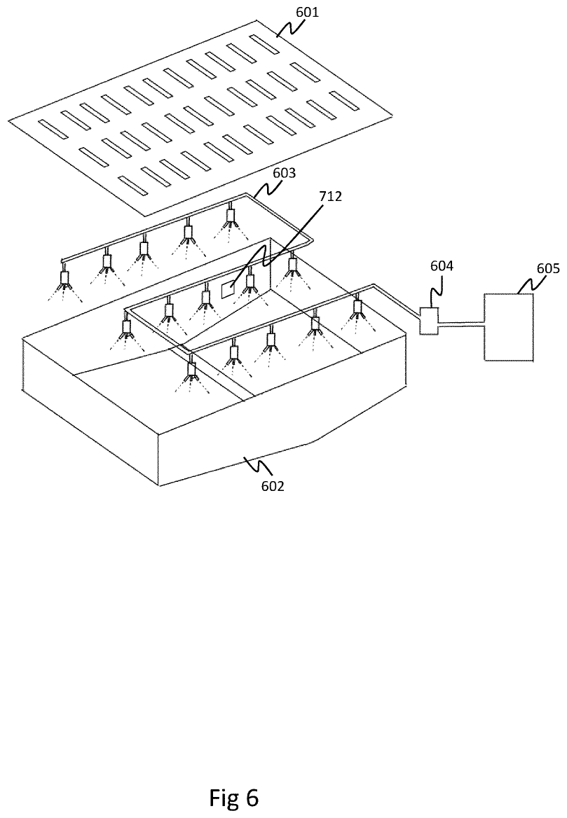

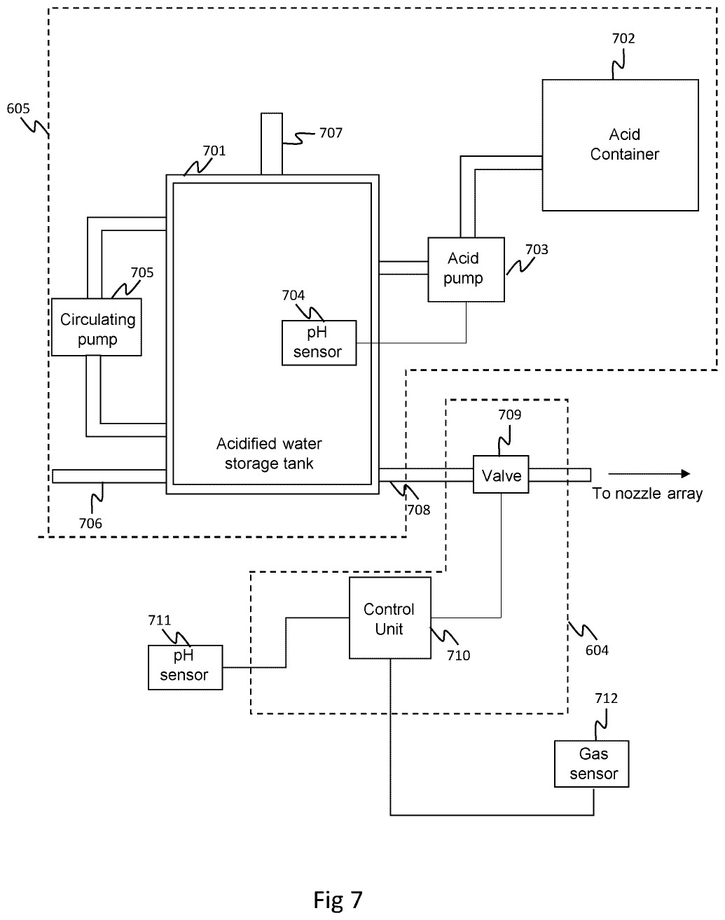

In accordance with a technique for treating waste, an otherwise conventional animal housing facility comprising an effluent pit into which waste from animals housed in the facility is collected, is adapted to include a spraying system which sprays the animal waste collected in the effluent pit with acidified water. An example embodiment is described further with reference to FIGS. 6 and 7.

FIG. 6 provides a simplified schematic diagram providing an exploded view depicting the positioning of a spraying system within an animal enclosure in accordance with a technique for treating waste. Such a spraying system can be retrofitted to an animal enclosure or incorporated in an animal enclosure as it is being constructed.

FIG. 6 shows the grated floor 601 of an animal enclosure which is positioned over an effluent pit 602. Waste from animals housed in the animal enclosure passes through grates of the grated floor 601 and collects in the effluent pit 602.

Suspended beneath the grated floor 601 is a nozzle array 603 configured in use to spray acidified water on effluent collected in the effluent pit 602. The nozzle array 303 is connected to a flow control arrangement 604 which is configured to control the flow of acidified water to the nozzle array 603 from a storage tank arrangement 605. The nozzle array 603, flow control arrangement 604 and storage tank arrangement 605 collectively form a spraying system.

FIG. 7 provides a simplified schematic diagram depicting in more detail components of the flow control arrangement 604 and storage tank arrangement 605.

The storage tank arrangement 605 comprises an acidified water storage tank 701 in which acidified water is mixed and stored. The storage tank arrangement 605 further comprises an acid container 702 which is connected to the acidified water storage tank 701 via an acid pump 703. In use, the acid container contains a supply of acid, typically nitric acid.

The acid pump 703 is connected to a pH sensor 704 configured to detect the pH of the acidified water in the acidified water storage tank 701. The acidified water storage tank 701 further comprises a circulating pump 705 for mixing the acidified water stored in the acidified water storage tank 701 and a water inlet 706 from a water supply. The acidified water storage tank 701 further comprises a pressure relief valve 707 which is configured to prevent the pressure in the acidified water storage tank 701 exceeding a predetermined pressure limit.

The acidified water storage tank 701 comprises an outlet 708 which connects the acidified water storage tank 701 to a flow control valve 709 of the flow control arrangement 604 which controls the flow of acidified water stored in the acidified water storage tank 701 to the nozzle array 603. The flow control arrangement 604 further comprises a valve control unit 710 which is connected to the flow control valve 709 and controls its operation.

In typical implementations, the acidified water storage tank 701 will be located relative to (i.e. at a greater height) the nozzle array 603 to produce sufficient pressure so that the acidified water is sprayed from the nozzle array 603 simply by opening the flow control valve 709. However, in implementations where this is not the case, a flow control pump can be provided instead of, or in addition to, the flow control valve 709 to pump acidified water from the acidified water storage tank 701 to nozzle array 603.

The components of the spraying system are typically made from suitable corrosive resistant materials, for example stainless steel.

In use, the valve control unit 710 is configured to periodically activate the flow control valve 709 causing acidified water from the acidified water storage tank 701 to flow to the nozzles of the nozzle array 603 and acidified water to be sprayed on effluent collected in the effluent pit 602. The frequency with which spraying occurs and the duration of the spraying is controlled by the valve control unit 710.

This frequency and duration of each spraying operation can be set at any appropriate values. For example, the valve control unit 710 may be configured to control the flow control valve 709 to perform four spraying operations a day, each of a duration of 30 minutes.

As the skilled person will understand, the total volume of acidified water sprayed on an effluent pit in accordance with embodiments of the technique for treating waste will depend upon the volume of waste stored in the effluent pit and the type of waste. In a typical example, there may be a ratio of 1000:1 between the volume of waste and volume of acid added to water and then sprayed on the collected waste. Thus, for example, for every 1000 litres of collected waste, 1 litre of acid is mixed with water and sprayed on the waste as acidified water during a given spraying operation.

The valve control unit 710 can be provided by any suitable control device as are well known in the art, for example a PLC (programable logic controller), an “embedded” system including a programmable microprocessor or a suitable personal computer.

The valve control unit 710 typical includes input means (for example, a display with a keypad, keyboard and/or mouse; or a touchscreen) for enabling an operative to control the operating parameters of the valve control unit, for example the frequency and duration of the spraying operations.

The flow control arrangement 604 further receives input from a slurry pH sensor 711 and a gas sensor 712.

The slurry pH sensor 711 is typically located in the effluent pit 602 and is configured to detect the pH of the waste collected in the effluent pit 602 and send a corresponding sensor signal to the valve control unit 710. The gas sensor 712 is typically located in the vicinity of the effluent pit 602 (a typical location is shown in FIG. 6) and is configured to detect levels of harmful gasses including ammonia gas and send a corresponding sensor signal to the valve control unit 710.

As well as controlling the flow control valve 709 so that the nozzle array 603 periodically sprays waste stored in the effluent pit 602 as described above, the valve control unit 710 is also configured to control the flow control valve 709 so that acidified water is sprayed from the nozzle array 603 onto waste collected in the effluent pit 602 if the gas sensor 712 generates a sensor signal indicating that the ammonia gas concentration (and/or any other harmful gas that the gas sensor is configured to detect) has exceeded a predetermined level. The valve control unit 710 controls the flow control valve 709 to continuing spraying acidified water until the sensor signal from the gas sensor 712 indicates that the ammonia gas concentration has fallen below the predetermined level.

The valve control unit 710 is configured to suspend spraying operations if the signal from the pH sensor 711 indicates that the pH of the waste collected in the effluent pit 602 has reached a desired pH level.

As a spraying operation is conducted, the quantity of acidified water in the acidified water storage tank 701 will reduce. After a spraying operation has been completed, the acidified water storage tank 701 is replenished with water from the water inlet 706. This is typically done automatically by a suitable valve/pump arrangement (not shown) which can detect the level of fluid in the acidified water storage tank 701.

The pH of the acidified water is detected by the pH sensor 704 which sends a corresponding sensor signal to the acid pump 703. The acid pump 703 is configured to pump acid from the acid container 702 if the sensor signal from the pH sensor 704 indicates a pH level which is below the desired pH level. In typical embodiments, the pH level will be between 3 to 5.5. The circulating pump 705 is configured to continuously or periodically circulate the acidified water in the acidified water storage tank 701 to ensure that acid from the acid container 702 is mixed with water from the water inlet 706. The acid pump 703 will continue to add acid from the acid container 702 to the acidified water storage tank 701 until the sensor signal from the pH sensor 704 indicates that the acidified water in the acidified water storage tank 701 is at the desired the desired pH level.

The spraying system, thus arranged, ensures that the animal waste collected in the grated floor 601 is regularly sprayed with acidified water.

Along with reducing the harmful emissions to the atmosphere and removing harmful pollutants from the animal waste stored in the effluent pit 602, by spraying the animal waste with acidified water at the point that it is collected in an effluent pit (in situ), when the slurry that is formed by the animal waste is later removed for use as a crop fertiliser it is “pre-treated” which means that it need not be treated as it is spread/injected during fertilizing operations.

The skilled person will understand that examples of the technique for treating waste can be implemented in alternative ways to the specific example described with reference to FIG. 6. In particular, there are many potential ways in which a suitable spraying system can be incorporated within an animal facility to spray an effluent pit with acidified water. For example, rather than the automatic replenishment of the acidified water storage tank 701 described above (facilitated by fresh water being provided from water inlet 706 and acid pumped from the acid container 702 under the control of the acid pump 703), the acidified water could be manually mixed and manually replenished. Similarly, rather than automatically controlling spraying operations with the valve control unit 710, the flow control valve 709 could be manually controlled. Further, the spraying means by which the acidified water is sprayed on the animal waste collected in the effluent pit 602 could be provided by a single nozzle or any suitable arrangement of nozzles rather than the nozzle array 603 comprising multiple nozzles depicted in FIG. 6.

The skilled person will also understand that spraying systems in accordance with the technique for treating waste can be incorporated in any suitable facility comprising an effluent pit in which waste collects. For example, a spraying system in accordance with certain embodiments of the technique for treating waste could be incorporated in a milk creamery facility to treat waste sludge from creamery processes collected in one or more effluent tanks located in the facility.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. Contaminated air treatment system, comprising:
at least one enclosed space for containing a source of contaminated air;
a subterranean treatment tunnel comprising one or more openings connecting the treatment tunnel with the enclosed space, said subterranean treatment tunnel having located therein contaminated air treatment means;
air circulating means configured to drive contaminated air from the at least one enclosed space into the treatment tunnel via the one or more openings and through the air treatment means, said treatment means comprising at least a first treatment stage comprising a spraying arrangement configured in use to spray the contaminated air to entrap contaminant particles in a liquid/sludge residue, and
a treated air exhaust to exhaust treated air after it has passed through the treatment tunnel.

2. Contaminated air treatment system according to claim 1, wherein, said treatment tunnel is located at substantially the same level as a base region of the at least one enclosed space.

3. Contaminated air treatment system according to claim 2, wherein the treatment tunnel runs at least a part of the length of the at least one base region.

4. Contaminated air treatment system according to claim 3, wherein the one or more openings are positioned along the length of the tunnel adjacent to the at least one base region.

5. Contaminated air treatment system according to claim 4, comprising two enclosed spaces, wherein said treatment tunnel is located between the base regions of each enclosed space.

6. Contaminated air treatment system according to claim 5, wherein the openings comprise a first set of openings positioned along the length of the tunnel on a first side of the tunnel adjacent the first base region of the first enclosed space and a second set of openings positioned along the length of the tunnel on a second side of the tunnel adjacent the second base region of the second enclosed space.

7. Contaminated air treatment system according to claim 6, wherein each opening of the first set of openings is positioned adjacent to a corresponding opening of the second set of openings, thereby inducing a turning effect in the air in the treatment tunnel in use.

8. Contaminated air treatment system according to claim 7, wherein each opening comprises a louver comprising at least one air-flow control slat which pivots about a lower edge to control an opening amount.

9. Contaminated air treatment system according to claim 2, wherein the base region or each base region comprises an effluent pit.

10. Contaminated air treatment system according to claim 1, wherein the one or more openings are adjustable to control an amount of contaminated air entering the treatment tunnel, thereby enabling a speed of the air in the treatment tunnel to be controlled.

11. Contaminated air treatment system according to claim 10, further comprising a pressure sensor and an actuator, said pressure sensor located in the treatment tunnel and configured to send a pressure senor signal to the actuator, said actuator coupled to the one or more openings and configured to control an opening amount of each of the one or more openings responsive to the pressure senor signal from the actuator.

12. Contaminated air treatment system according to claim 11, wherein the actuator is configured to control the one or more openings to maintain a predetermined air speed in the treatment tunnel.

13. Contaminated air treatment system according to claim 1, wherein the air treatment means comprises a second treatment stage comprising a spraying arrangement configured in use to spray the air from the first stage with an odorant.

14. Contaminated air treatment system according to claim 1, wherein the air circulating means comprises a fan bank comprising one or more fans located in the treatment tunnel.

15. Contaminated air treatment system according to claim 14, wherein the fan bank is positioned between the first treatment stage and the second treatment stage.

16. Contaminated air treatment system according to claim 1, wherein the treatment tunnel further comprises a waste tank to collect the liquid/sludge residue.

17. Contaminated air treatment system according to claim 1, wherein the treated air exhaust is located to exhaust the treated air at ground level.

18. Contaminated air treatment system according to claim 1, wherein a final filtration stage is located on an outlet side of the treated air exhaust to further filter the treated air.

19. Contaminated air treatment system according to claim 18, wherein the final filtration stage comprises a filtration compound made from wood and/or bark chippings.

20. Contaminated air treatment system according to claim 1, wherein the at least one enclosed space comprises at least one vent opening the at least one enclosed space to the outside environment, wherein said air circulating means is configured to draw fresh air in through the at least one vent thereby ventilating the at least one enclosed space.

21-40. (canceled)
